(19) United States
(12) Patent Application Publication
(10) Pub. No.: US 2008/0125751 A1
(43) Pub. Date: May 29, 2008

(54) TEMPERATURE COMPENSATION FOR ENZYME ELECTRODES

(75) Inventors: Todd Fjield, Laguna Hills, CA (US); Michael Higgins, Huntington Beach, CA (US); Kenneth Curry, Oceanside, CA (US); Patrick Carlin, Dana Point, CA (US)

Correspondence Address: EDWARDS LIFESCIENCES CORPORATION, LEGAL DEPARTMENT, ONE EDWARDS WAY, IRVINE, CA, 92614, US

(73) Assignee: Edwards Lifesciences Corporation, Irvine, CA (US)

(21) Appl. No.: 11/940,037

(22) Filed: Nov. 14, 2007

Related U.S. Application Data
(63) Continuation-in-part of application No. 10/046,890, filed on Jan. 14, 2002, now abandoned.
(60) Provisional application No. 60/859,586, filed on Nov. 16, 2006.

(51) Int. Cl. A61M 25/00; C12Q 1/00

(52) U.S. Cl. 604/523; 205/777.5

(57) ABSTRACT

A temperature compensation method for an enzyme electrode by measuring an operating temperature of the enzyme electrode, measuring the current generated by the enzyme electrode determining a deviation in measurement between the current generated and a reference current at the operating temperature, determining an enzyme concentration corresponding to the measured current, and calibrating the enzyme concentration to compensate for the deviation in measurement.

### CLAIM OF PRIORITY UNDER 35 §119

The present application claims priority from U.S. Provisional Patent Application No. 60/859,586, filed Nov. 16, 2006, entitled “TEMPERATURE COMPENSATION FOR ENZYME ELECTRODES,” which is assigned to the assignee hereof and hereby expressly incorporated by reference herein.

### BACKGROUND OF THE INVENTION

1. Field of the Invention

The invention relates generally to enzyme electrodes. More particularly, the invention relates to temperature compensation for enzyme electrodes.

2. Description of Related Art

When diabetics control their blood sugar (glucose), they are more likely to live and stay healthy. They may monitor and test for glucose in the blood using a prior art glucose monitoring system, such as an amperometric glucose detector. The glucose monitoring system is designed to control amperometric biosensors in a static and stable environment, such as a medical laboratory. The amperometric biosensors may be coated with chemicals, such as glucose oxidase, dehydrogenase or hexokinase, which combine with glucose in the blood sample. Some sensors measure the amount of current generated by the sensor in the blood sample, while others measure how much light reflects from it. These measurements are further analyzed and quantified by the glucose monitoring system to determine the glucose level in the blood sample.

Recently, new sensors have been introduced into the market that can be inserted percutaneously into subcutaneous tissue. These sensors provide continuous, or near continuous, readings of glucose concentration, thereby allowing patients to better manage their glucose levels.

The biosensors are calibrated to provide actual measurements at a specific temperature. FIG. 1 is a graph illustrating the relationship between the glucose level in the blood sample and the current measured from the biosensors at varying temperatures. The measurements obtained from the biosensors are dependent on the temperature of the surroundings. If the temperature of the surroundings changes, an error occurs in the measurements. An increase in temperature increases the slope of the curve, while a decrease in temperature decreases the slope of the curve. If the slope increases, the computed glucose level is lower than the actual glucose level. In contrast, if the slope decreases the computed glucose level is higher than the actual glucose level. Hence, a change in temperature of the surroundings provides an error in the computed glucose level.

FIG. 2 is a graph illustrating current change as a function of temperature. Data from a prior art glucose monitoring system was taken at four different glucose concentrations over a temperature range of 32° C. to 41° C. The current was normalized to 1 at 37° C. As shown for the different glucose concentrations, an increase in temperature increases the current measured from the biosensors, thereby providing an inaccurate measurement of the glucose level in the blood. The resulting error is illustrated in the Clark Error grid of FIG. 3. The grid shows how the glucose measurements, without temperature compensation, compare to the true glucose concentration values.

As is well known in the art, Zone A represents clinically accurate measurements. Zone B represents measurements deviating from the reference glucose level by more than 20% but would lead to benign or no treatment, Zone C represents measurements deviating from the reference glucose level by more than 20% and would lead to unnecessary corrective treatment errors. Zone D represents measurements that are potentially dangerous by failing to detect and treat blood glucose levels outside of desired target range. Finally, Zone E represents measurements resulting in erroneous treatment. As shown in the Clark Error grid of FIG. 3, some of the error measurements were close to the Zone B, thereby deviating from the reference by more than 20%. Hence, when no temperature compensation is employed there are large errors.

There are many factors that can affect a change in the temperature surrounding the sensor. Since sensors are inserted in the human body, via a catheter, the temperature of the body may affect the sensor readings. The body temperature may be higher or lower than the temperature at which the sensors were calibrated. The sensors may also be affected by the room temperature prior to insertion in the human body. Furthermore, the infusion of fluid through a lumen in the catheter can have an affect on the sensor's measurements. The fluid may have a different temperature from the human body, and accordingly, would affect the sensor's readings during fluid infusion.

Depending on the location of the sensor and the configuration of the device in which the sensor is located, temperature changes may cause the current produced by the sensor to change for the same glucose concentration, thereby invalidating the calibration curves. This may cause the accuracy of these sensors to be unacceptable for clinical use and perhaps dangerous for guiding therapy.

Past solutions include withdrawing a sample of blood and measuring the glucose level in an isolate static environment with constant temperature. Another prior art method includes withdrawing a sample of blood across a sensor and recirculating the blood back to the patient. These solutions do not compensate for the temperature changes; rather, they seek to avoid the possibility of temperature changes.

With an increasing demand for improved glucose monitoring systems, there remains a need in the art for temperature compensation for sensor electrodes to provide reliable measurements despite a change in surrounding temperature.

### SUMMARY OF THE INVENTION

The present invention fills this need by providing a temperature compensation method for an enzyme electrode by measuring an operating temperature of the enzyme electrode, measuring the current generated by the enzyme electrode, determining a deviation in temperature between the operating temperature and the reference temperature, determining a glucose concentration corresponding to the measured current at the operating temperature, and compensating the glucose concentration measurement for the deviation in temperature.

In one embodiment, temperature compensation may be achieved by using a calibration curve that corrects for the variation in the current produced due to a temperature change. For an enzyme electrode with linear or nearly linear characteristics, the glucose concentration with temperature compensation=slope·current·e^{T}^{_{coeff}}^{(T}^{_{cal}}^{-T)}. “Absolute” or “relative” calibration curves may be determined for an electrode with nonlinear characteristics.

### BRIEF DESCRIPTION OF THE DRAWINGS

The exact nature of this invention, as well as the objects and advantages thereof, will become readily apparent from consideration of the following specification in conjunction with the accompanying drawings in which like reference numerals designate like parts throughout the figures thereof and wherein:

FIG. 1 is a graph illustrating the relationship between the glucose level in the blood sample and the current measured from the biosensors at varying temperatures.

FIG. 2 is a graph illustrating current change as a function of temperature at several glucose concentrations.

FIG. 3 is a Clark Error Grid illustrating prior art glucose measurements, without temperature compensations in relation to true glucose concentration values.

FIG. 4 illustrates a catheter with a temperature element included for the purpose of temperature compensation.

FIG. 5 is a cross-sectional view of the catheter of FIG. 4 along line 5-5.

FIG. 6 is a cross-sectional view of the catheter of FIG. 4 along line 6-6.

FIG. 7 is a graph illustrating the change in temperature as a function of time.

FIG. 8 is a graph illustrating the glucose concentration measurement, with and without temperature compensation, relative to true glucose levels as a function of time, when the sensor is subjected to the temperature variation as shown in FIG. 7.

FIG. 9 is a Clark Error Grid illustrating glucose measurements, with temperature compensation, in relation to true glucose concentration values.

FIG. 10 is a cross-sectional view of the sensor with a temperature compensation element.

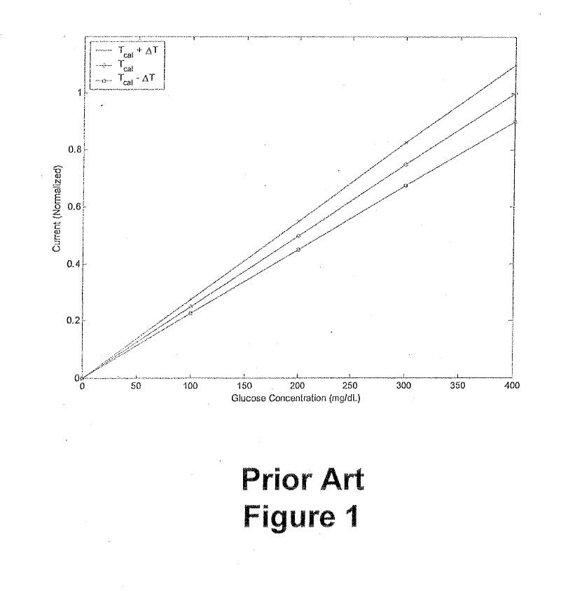

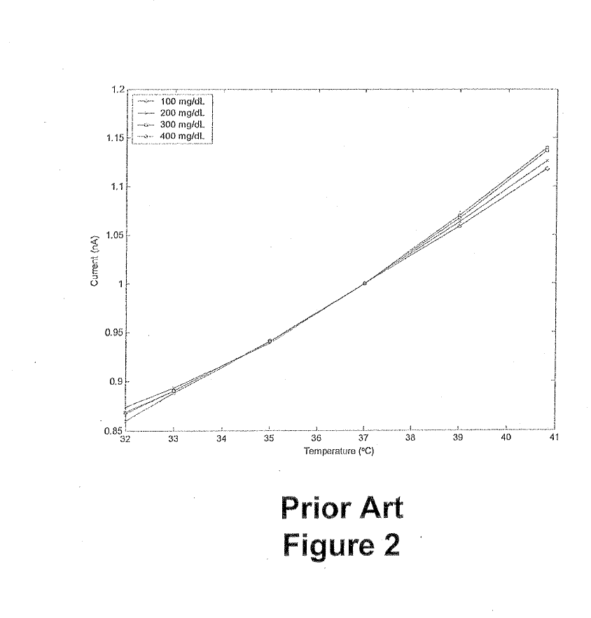

### DETAILED DESCRIPTION

A sensor electrode operable in an environment with varying temperature is provided. The sensor provides glucose measurements with acceptable accuracy for clinical setting, specifically to guide therapy. The sensor may be used in an access device, such as a catheter, for both venous and arterial environments. The catheter may be configured to allow for the infusion of fluid. The fluid may infuse into the body at a temperature different from the body temperature.

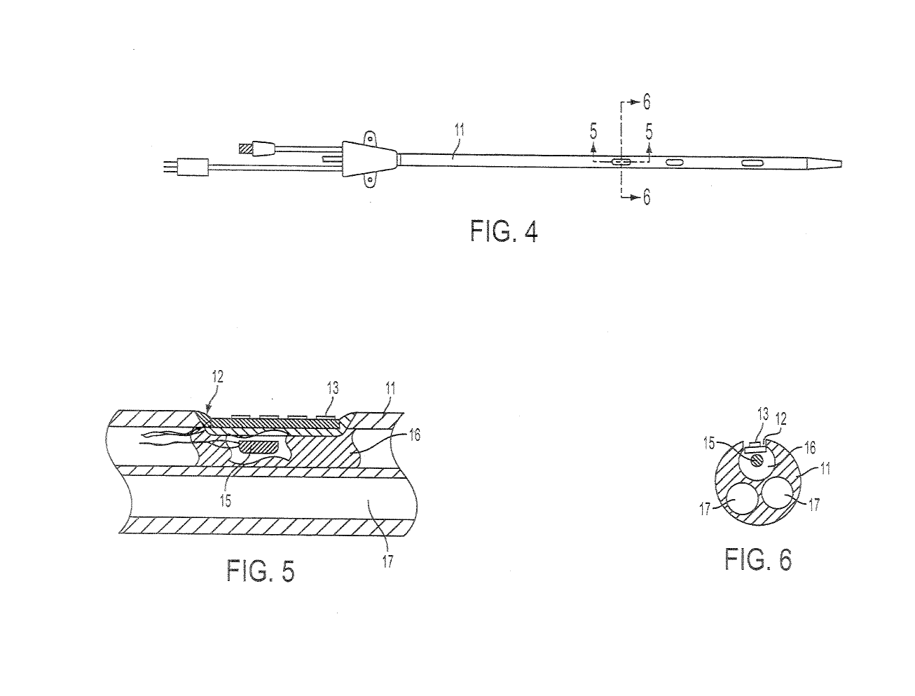

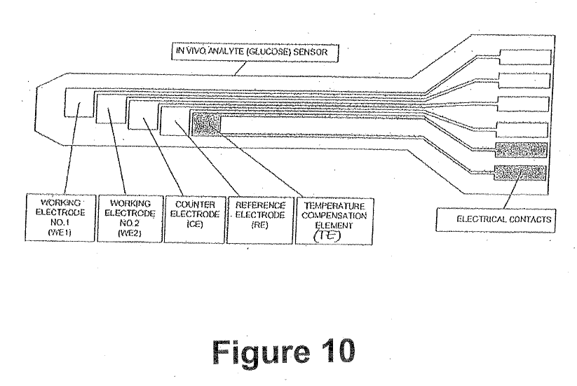

FIG. 4 illustrates an example of a catheter 11 (e.g., a glucose monitoring catheter). FIG. 5 is a cross-sectional view of the catheter 11 of FIG. 4. FIG. 10 is a cross-sectional view of a sensor (e.g., an enzyme electrode or a glucose electrode or sensor) with a temperature sensing device or temperature compensation element 15. The catheter 11 has at least one opening 12 that exposes one or more sensor electrodes 13. In an embodiment, underneath the sensor electrodes 13 is a temperature sensing device, such as a thermistor 15, held in place by adhesive or filling material 6, as shown in FIG. 6. The catheter 11 also has one or more pathways, such as lumens 17, along its length for infusion of fluid in the blood. The flow of fluid in pathways 17 of the catheter 11 can have an affect on the sensor's measurements. The fluid may have a different temperature from the human body, and accordingly, would affect the sensor 13 readings during fluid infusion.

The current produced by the sensor electrode 13 for a given analyte concentration is based on a number of factors. For example, it depends on the concentration of enzymes and the diffusion rates through the membrane containing or encapsulating the electrodes, such as a polyurethane, hydro-polymer or gel membrane. The turnover rate of the enzymes and the diffusion rates through the membrane are typically temperature dependent. While the purpose of the sensor electrode 13 is to produce a known magnitude of current for a known concentration of an analyte, a small temperature variation can introduce an error in the measurement. Typically, errors resulting from temperature variation range from 2 to 7%.

One way to mitigate the error introduced by temperature variation is to control the temperature of the sensor 13 and/or solution containing the analyte of interest, such that the temperature remains constant. However, when the sensor is integrated into a catheter 11, controlling the temperature of the sensor 13 and/or solution is not feasible. For example, body temperature changes or a temperature and/or rate of an infusion fluid would affect the sensor reading. Accordingly, temperature compensation is necessary to obtain accurate measurements. The catheter 11 may be an intravascular catheter.

The temperature compensation or sensing element 15 (e.g., a thermistor or a silver trace or any device whose resistance changes with changing temperature) may be attached to the sensor 13, located adjacent to the sensor 13, co-located on the same plane or membrane as the sensor 13, integrated into the sensor 13 itself, attached to a device in which the sensor 13 is located, placed in the vicinity of the sensor 13, placed at a location that is representative of the temperature around the sensor 13, or placed in a location that tracks the temperature variation around the sensor 13. The temperature sensing element 15 and/or the sensor 13 may be positioned within the catheter 11. The temperature sensing element 15 measures temperature at the sensor 13 to compensate for blood or infusates traveling through the catheter 11. In one embodiment, the temperature sensing element 15 may be configured or positioned so that it can measure the temperature of the sensor 13 or a change in temperature due to an external condition (e.g., body temperature) or an internal condition (e.g., infusates). The infusate rate may also need to be calculated during the internal condition. In one embodiment, the temperature sensing element 15 directly measure the temperature of the sensor 13 that is in contact with the blood stream.

Preferably, the temperature sensing element 15 may be insulated from the infusion fluid using insulating structures, as disclosed in U.S. Pub. No. 2002/0128568, and incorporated herein by reference. Various insulating lumens 17 and insulating members may be used to insulate the temperature sensing element 15 from the infusion fluid, which might otherwise degrade the accuracy of the temperature measurement.

Temperature compensation may be achieved by using a temperature compensation element that corrects/calibrates for the error in the current measurement due to a temperature change. Under predetermined operating conditions, the effect of temperature on the calibration curve of the temperature compensation element may be an increase in the first order term at higher temperatures and a change in the offset. For electrodes 13 with linear or nearly linear characteristics, the first order term is the slope. Hence, the temperature compensation for electrodes 13 with linear or nearly linear characteristics may be expressed in the following form:

Correction Factor=ΔT·T_{coeff}·slope (1)

where,

ΔT is the change in temperature from the temperature at which the electrode 13 was calibrated;

T_{coeff} is the temperature coefficient (change in slope per degree); and

slope is the change in analyte concentration divided by the change in current.

Equation (1) holds true for glucose electrodes 13 with linear or nearly linear characteristics where there is no infusion of fluid through the catheter over the temperature range in which the correction factor remains linear or nearly linear with temperature. However, a calibration curve may also be used for a sensor 13 with non-linear characteristics, where fluid is infused into the body through lumen 17 in the catheter 11.

An “absolute” or “relative” calibration curve may be determined for glucose electrodes 13 with non-linear characteristics. For an “absolute” calibration curve, a correction factor or calibration curve is ascertained at specific measured temperatures, whereas for a “relative” calibration curve, a correction factor is determined based on a temperature change from the temperature at which the electrode 13 was calibrated and/or another reference temperature.

According to a temperature compensation method for glucose electrodes with linear or non-linear characteristics, the temperature of the area or solution surrounding the sensor 13 or the temperature of a device to which the sensor is attached is measured by the temperature sensing element 15. Based on previous measurements, an individual calibration curve at the measured temperature is predetermined. As the temperature changes, due to an infusion of fluid, for example, various calibration curves may be substituted, such that each calibration curve reflects the current produced as a function of analyte concentration at the measured temperature.

According to another temperature compensation method for glucose with linear or non-linear characteristics, the temperature deviation from the temperature at which the electrodes 13 was calibrated is measured by a temperature sensing element 15. Based on this deviation, calibration curves may be substituted, such that each calibration curve reflects the current produced as a function of analyte concentration at the measured temperature deviation.

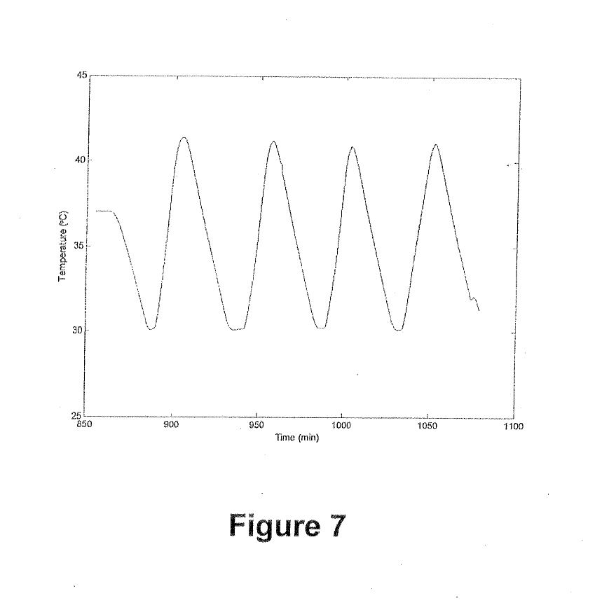

To better demonstrate the effect of calibration curves on glucose measurements, an exemplary in vitro test is described with and without temperature compensation. The temperature of the area or solution surrounding the sensor 13 or the temperature of a device the sensor 13a is attached was varied from 30° C. to 42° C. over time, as shown in FIG. 7. After a predetermined period, the glucose concentration was increased by about 100 mg/dL for about every 40 minutes.

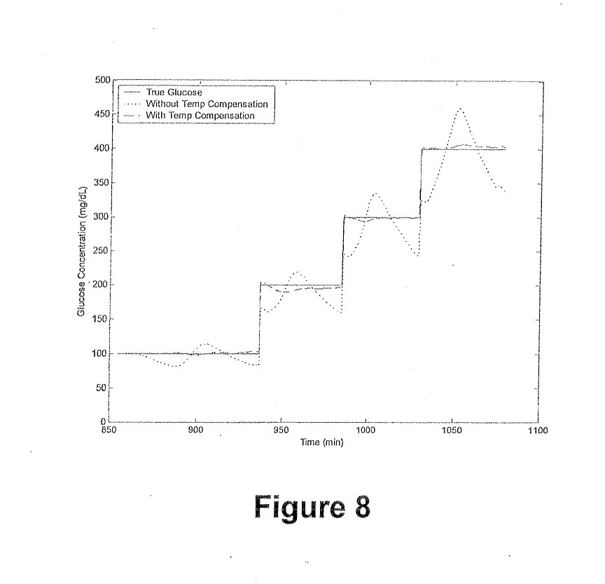

FIG. 8 is a graph illustrating the change in glucose concentration over a period of time. As shown in FIG. 8, the solid line illustrates the true glucose concentration at a specific time, the dotted line represents the measured glucose concentration without temperature compensation, and the dashed line represents the measured glucose concentration with temperature compensation. The temperature compensation used in FIG. 8 was in the form:

glucose concentration=slope·current·e^{T}^{_{coeff}}^{(T}^{_{cal}}^{-T)} (2)

where, slope is the change in glucose concentration divided by the change in current;
current is the current generated by the electrode 13;
T_{coeff} is the temperature coefficient of the sensor(s);
T_{cal} is the temperature at which the electrode 13 was calibrated; and
T is the temperature of the electrode 13 measured by the temperature sensing element 15.

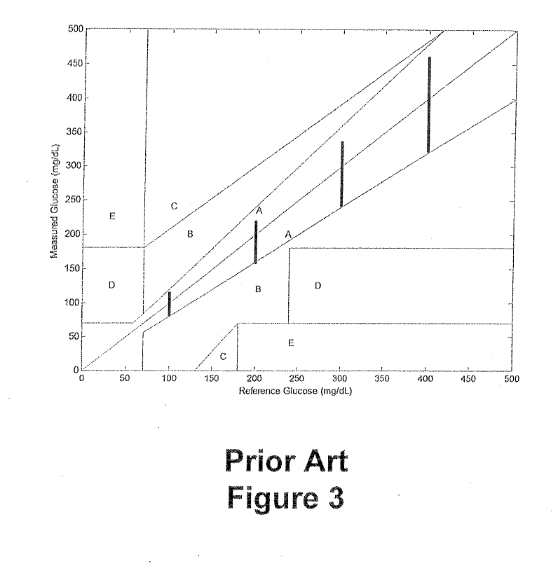

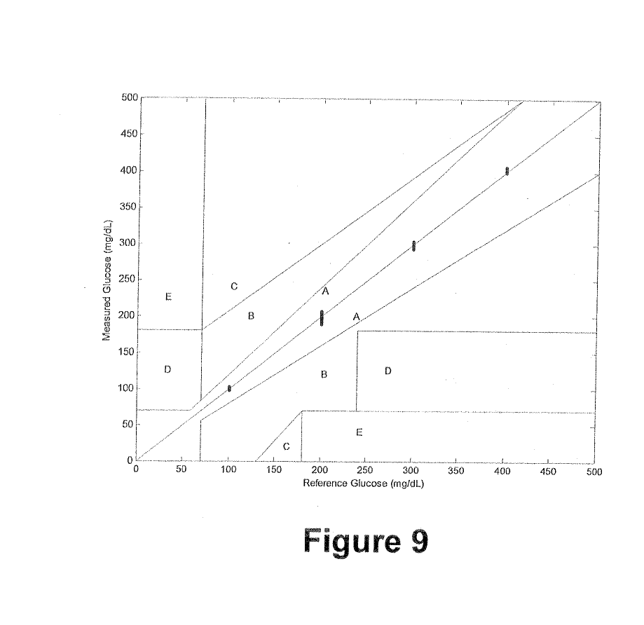

Without temperature compensation, there are large errors in the measured glucose values. However, with temperature compensation using equation (2), the measured glucose values line up relatively close to the true glucose values. A Clark Error grid, illustrated in FIG. 9, shows how the glucose measurements, with temperature compensation, compare to the true glucose concentration values. The Clark Error grid of FIG. 9 shows significantly less error in measured glucose concentration, when compared to the Clark Error grid of FIG. 3. The measured glucose concentration with temperature compensation is clinically accurate (Zone A) with measurements close to the reference glucose level.

While certain exemplary embodiments have been described and shown in the accompanying drawings, it is to be understood that such embodiments are merely illustrative of and not restrictive on the broad invention, and that this invention not be limited to the specific constructions and arrangements shown and described, since various other changes, combinations, omissions, modifications and substitutions, in addition to those set forth in the above paragraphs, are possible. Those skilled in the art will appreciate that various adaptations and modifications of the just described preferred embodiment can be configured without departing from the scope and spirit of the invention. Therefore, it is to be understood that, within the scope of the appended claims, the invention may be practiced other than as specifically described herein.

For example, the temperature compensation was described in the context of sensor 13. A person skilled in the art would understand that the temperature compensation of the invention may be applied to other enzyme electrodes and/or other biosensors affected by temperature change.

While certain embodiments were described in the context of using one temperature sensing element 15 to measure the temperature of the sensor, those skilled in the art would appreciate the use of a plurality of temperature sensing elements 15 that would aid in obtaining a calibration curve for different operating conditions. For example, two temperature sensing elements may be used to measure temperature: one temperature sensing element measures the body temperature (T1) while the second temperature sensing element measures the temperature (T2) of the infusion fluid. The temperature results may be calibrated and correlated to obtain an analyte calibration curve that is compensated by a function of temperature (T1) and temperature (T2).

Additionally, while the examples included herein illustrate temperature correction factors dependent only on a constant temperature coefficient and temperature, those skilled in the art would recognize a temperature coefficient and/or correction factor that was dependent on the estimated or measured glucose concentration, oxygen tension, and/or pH, for example, as being part of the same invention.

What is claimed is:

1. An apparatus for compensating for temperature comprising:
a catheter having a generally tubular body defining an opening and a lumen positioned adjacent to the opening;
a sensor positioned in the opening for producing a current; and
a temperature sensing device positioned in the lumen for determining a temperature of an area adjacent to the sensor and for compensating for an output of the sensor.

2. The apparatus of claim 1 wherein the catheter is selected from a group consisting of a glucose monitoring catheter and an intravascular catheter.

3. The apparatus of claim 1 wherein the sensor is selected from a group consisting of an enzyme electrode and a glucose electrode.

4. The apparatus of claim 1 further comprising a material used to hold the temperature sensing device in the lumen.

5. The apparatus of claim 1 wherein the temperature sensing device is a thermistor.

6. The apparatus of claim 1 further comprising a pathway positioned adjacent to the lumen for passing fluid.

7. The apparatus of claim 1 further comprising a membrane for containing the sensor.

8. The apparatus of claim 7 wherein the membrane is selected from a group consisting of a polyurethane membrane, a hydro-polymer membrane and a gel membrane.

9. A catheter for insertion into a body, the catheter comprising:
a sensor for generating a signal in response to an analyte concentration in the body; and
a temperature compensation element for determining a temperature of an area adjacent to the sensor and for compensating for an output of the sensor.

10. The catheter of claim 9 wherein the sensor is selected from a group consisting of an enzyme electrode and a glucose electrode.

11. The catheter of claim 9 wherein the temperature compensation element is a thermistor.

12. The catheter of claim 9 further comprising a membrane for containing the sensor.

13. The catheter of claim 12 wherein the membrane is selected from a group consisting of a polyurethane membrane, a hydro-polymer membrane and a gel membrane.

14. An apparatus for compensating for temperature comprising:
a generally tubular catheter body defining an opening;
a sensor positioned in the opening for producing a current in response to an analyte concentration; and
a temperature sensing device positioned adjacent to the sensor for determining a temperature of an area adjacent to the sensor and for compensating for an output of the sensor.

15. The apparatus of claim 14 wherein the sensor is selected from a group consisting of an enzyme electrode and a glucose electrode.

16. The apparatus of claim 14 further comprising a material used to hold the temperature sensing device in the opening.

17. The apparatus of claim 14 wherein the temperature sensing device is a thermistor.

18. The apparatus of claim 14 further comprising a pathway positioned adjacent to the temperature sensing device for passing fluid.

19. The apparatus of claim 14 further comprising a pathway positioned adjacent to the sensor for passing fluid.

20. The apparatus of claim 14 further comprising a membrane for coating the sensor.

21. The apparatus of claim 20 wherein the membrane is selected from a group consisting of a polyurethane membrane, a hydro-polymer membrane and a gel membrane.

22. A method for temperature compensation of an electrode comprising:
measuring a reference current;
measuring an electrode current received from the electrode;
determining a difference between the reference current and the electrode current;
determining an enzyme concentration corresponding to the electrode current; and
adjusting the enzyme concentration based on the difference between the reference current and the electrode current.

23. The method of claim 22 further comprising measuring an operating temperature of the electrode.

24. The method of claim 22 wherein the enzyme concentration is determined using the formula:
glucose concentration=slope·current·e^{T}^{_{coeff}}^{(T}^{_{cal}}^{-T)}
where,
slope is a predetermined characteristic of the electrode;
current is the current generated by the electrode;
T_{coeff} is the temperature coefficient of the electrode;
T_{cal} is the temperature at which the electrode was calibrated; and
T is the operating temperature of the electrode.

25. A temperature compensation method for an enzyme electrode comprising:
measuring an operating temperature of the enzyme electrode;
measuring the current generated by the enzyme electrode;
determining a deviation in measurement between the measured current and a reference current at the operating temperature;
determining an enzyme concentration corresponding to the measured current; and
calibrating the enzyme concentration to compensate for the deviation in measurement using the relation:
glucose concentration=slope·current·e^{T}^{_{coeff}}^{(T}^{_{cal}}^{-T)}
where,
slope is a predetermined characteristic of the enzyme electrode;
current is the current generated by the enzyme electrode;
T_{coeff} is the temperature coefficient of the enzyme electrode;
T_{cal} is the temperature at which the enzyme electrode was calibrated; and
T is the operating temperature of the enzyme electrode.
